(19) United States
(12) Plant Patent Application Publication
(10) Pub. No.: US 2015/0296677 P1
(43) Pub. Date: Oct. 15, 2015

(54) Petunia plant named 'DRAY307'

(50) ;

(71) Applicant: Gavriel Danziger, Moshav Mishmar Hashiva (IL)

(72) Inventor: Gavriel Danziger, Moshav Mishmar Hashiva (IL)

(21) Appl. No.: 13/999,987

(22) Filed: Apr. 11, 2014

(51) Int. Cl. A01H 5/00

(52) U.S. Cl. PLT/356.1

(57) ABSTRACT

A new and distinct Petunia cultivar named ‘DRAY307’ is disclosed, characterized by abundant large yellow flowers on a mounded ball shaped plant, very branched plants of medium vigor. The new variety is an Petunia, normally produced as an outdoor garden or container plant.

### LATIN NAME OF THE GENUS AND SPECIES

Petunia sensu wijsman

### VARIETY DENOMINATION

‘DRAY307’

### BACKGROUND OF THE INVENTION

The new Petunia cultivar is a product of a planned breeding program conducted by the inventor, Gavriel Danziger, in Moshav Mishmar Hashiva, Israel. The objective of the breeding program was to produce new Petunia varieties for ornamental commercial applications. The cross resulting in this new variety was made during January of 2011.

The seed parent is the unpatented, proprietary variety referred to as Petunia sensu wijsman. ‘cv. 8-1255’.The pollen parent is the unpatented, proprietary variety referred to as Petunia sensu wijsman ‘cv. 10-3970. The new variety was discovered in December of 2011 by the inventor in a group of seedlings resulting from the 2011 crossing, in a research greenhouse in Moshav Mishmar Hashiva, Israel.

Asexual reproduction of the new cultivar was performed by vegetative cuttings. This was first performed at a research greenhouse in Moshav Mishmar Hashiva, Israel in January of 2012 and has shown that the unique features of this cultivar are stable and reproduced true to type in more than 25 successive generations.

### SUMMARY OF THE INVENTION

The cultivar ‘DRAY307’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘DRAY307’ These characteristics in combination distinguish ‘DRAY307’ as a new and distinct Petunia cultivar:

1. Mounded, ball-shaped.

2. Very branched with medium vigor.

3. Flower color is yellow.

4. Flower size is large.

5. Abundant flowering.

Plants of the new cultivar ‘DRAY307’ are similar to plants of the seed parent, Petunia sensu wijsman ‘cv. 8-1255’ in most horticultural characteristics, however, plants of the new cultivar ‘DRAY307’ differs in the following;

1. Mounded, ball shape, compared to the semi upright shape of seed parent.

2. Larger far more branched plant.

3. Different flower color yellow compared to cream color of seed parent.

4. Larger flower size.

5. Greater amount of flowers.

Plants of the new cultivar ‘DRAY307’ are similar to plants of the pollen parent; Petunia sensu wijsman ‘cv. 10-3970’ in most horticultural characteristics, however, plants of the new cultivar ‘DRAY307’ differs in the following;

1. Ball shaped habit.

2. Smaller and less vigorous and branched plant

3. Flowers are yellow; flowers of pollen parent are greenish-yellow.

4. Smaller flower size.

5. Less abundant flowering.

### COMMERCIAL COMPARISON

Plants of the new cultivar ‘DRAY307’ are comparable to the variety Petunia ‘DANRAY1’ U.S. Plant Pat. No. 20,974. The two Petunia varieties are similar in most horticultural characteristics; however, the new variety ‘DRAY307’ differs in the following:

1. Smaller flower size.

2. Yellow flowers. The comparator has white flowers.

3. Less vigor.

4. Shorter branches.

Plants of the new cultivar ‘DRAY307’ can also be compared to the commercial variety Petunia ‘DANRAY3’, unpatented. These varieties are similar in most horticultural characteristics; however ‘DRAY307’ differs in the following:

1. Smaller flower size.

2. Yellow flowers. The comparator has light purple flowers with dark purple veins.

3. Less vigor.

4. Shorter branches.

### BRIEF DESCRIPTION OF THE PHOTOGRAPHS

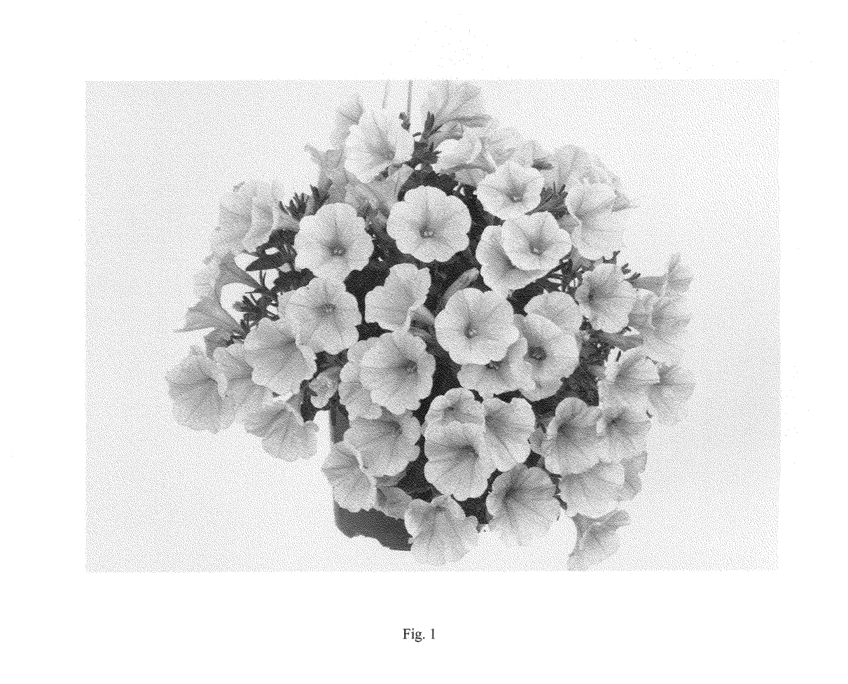

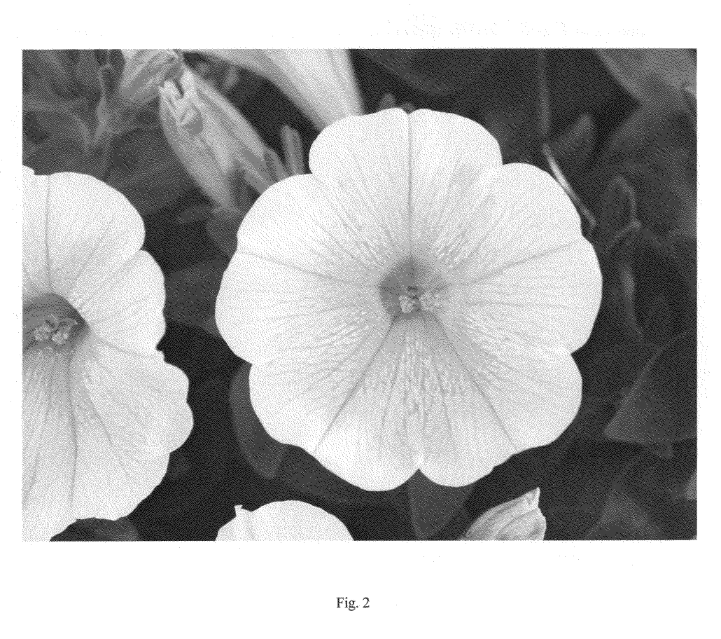

The accompanying photograph in FIG. 1 illustrates in full color a typical plant of ‘DRAY307’ grown in a greenhouse, in a 13 cm pot. Age of the plant photographed is approximately 3 months from a rooted cutting. FIG. 2 illustrates in full color a close up of a typical bloom of ‘DRAY307’. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Mini Colour Chart 2005 except where general terms of ordinary dictionary significance are used. The following observations and measurements describe ‘DRAY307’ plants grown in a greenhouse, in Moshav Mishmar Hashiva, Israel. The growing temperature ranged from 20° C. to 35° C. during the day and from 17° C. to 23° C. during the night. General light conditions are bright, normal sunlight. Measurements and numerical values represent averages of typical plant types.
Botanical classification: Petunia sensu wijsman ‘DRAY307’
Propagation:
   Time to initiate roots.—About 10 to 14 days at approximately 22° C.
   Root description.—Fibrous.
Plant:
   Age of plant described.—Approximately 60 days from a rooted cutting.
   Growth habit.—Mounded.
   Pot size of plant described.—13 cm.
   Height.—To top of flowers: About 22 cm.
   Plant spread.—About 50 cm.
   Growth rate.—Medium.
   Branching characteristics.—Very branched.
   Length of primary lateral branches.—About 25 cm.
   Diameter of lateral branches.—About 0.3 cm.
   Quantity of primary lateral branches.—8.
   Characteristics of primary lateral branches.—Form: Cylindrical Diameter: About 0.3 cm. Color: RHS Yellow-Green 144 A Texture: Pubescent Strength: Good.
   Internode length.—About 1.5 cm.
Foliage:
   Leaf.—Arrangement: Opposite Quantity: Approximately 20 per branch. Average Length: 5.5 cm. Average Width: 3.0 cm. Shape of blade: Elliptic Apex: Acute Base: Acute Margin: Entire Texture of upper and lower surfaces: Velvety Aspect: Mainly flat, 90 degree angle of attachment. Color: Young foliage upper side: RHS Green 137 B Young foliage under side: RHS Green 137 C Mature foliage upper side: RHS Green 137 B Mature foliage under side: RHS Green 137 C Venation: Type: Pinnate Venation color upper side: RHS Green 137 C Venation color under side: RHS Green 137 D Petiole: Length: About 0.7 cm. Diameter: About 0.2 cm. Color: RHS Green 137 B Texture: Velvety.
Flower:
   Natural flowering season.—Spring, Summer and Autumn.
   Days to flowering from rooted cutting.—about 30 days.
   Inflorescence and flower type and habit.—Axillary, single flower, Salverform shape, Erect habit.
   Rate of flower opening.—2 to 3 days from bud to fully opened flower.
   Flower longevity on plant.—3-6 days.
   Approximate quantity of flowers per plant.—About 60.
   Persistent or self-cleaning.—Self-cleaning.
   Bud.—Shape: Tubular Length: About 4.0 cm Diameter: About 1.0 cm Color: RHS Yellow-Green 154 A.
   Flower size.—Diameter: About 6 cm. Flower tube length: About 3 cm. Flower tube diameter at distal end: 1 cm. Flower tube diameter at proximal end: 0.3 cm.
   Petals.—Length from throat: About 3 cm. Width: About 3 cm. Quantity: 5 Texture: Velvety Apex: Blunt Margin: Wavy.
   Color.—Arrangement: Opposite Quantity: Approximately 20 per branch. Average Length: 5.5 cm. Average Width: 3.0 cm. Shape of blade: Elliptic Apex: Acute Base: Acute Margin: Entire Texture of surfaces: Velvety Pubescence: Yes. Color: Young foliage upper side: RHS Green 137 B Young foliage under side: RHS Green 137 C Mature foliage upper side: RHS Green 137 B Mature foliage under side: RHS Green 137 C Venation: Type: Pinnate Venation color upper side: RHS Green 137 C Venation color under side: RHS Green 137 D Petiole: Length: About 0.7 cm. Diameter: About 0.2 cm. Color: RHS Green 137 B Texture: Velvety.
Reproductive organs:
   Stamens.—Number: 5 Filament length: About 2.0 cm.
   Anthers.—Length: About 0.1 cm Shape: Rounded Color: RHS Yellow 11 B Pollen: Color: RHS Yellow 11 B Quantity: Plenty.
   Pistil.—Number: 1 Length: About 2.5 cm. Style: Length: About 2 cm. Color: RHS Yellow-Green 154 C Stigma: Shape: Rounded Color: RHS Green 143 C Ovary Color: RHS Green 143 B.
Other characteristics:
   Seeds and fruits.—About 50 brown, rounded seeds of about 0.5 mm diameter, per one brown, conical capsule.
   Disease/pest resistance.—Neither resistance nor susceptibility to normal diseases and pests of Petunia have been observed.
   Temperature tolerance.—Typical range 5° C.-40° C.

What is claimed is:

1. A new and distinct cultivar of Petunia plant named ‘DRAY307’ as herein illustrated and described.
